(19) United States
(12) Patent Application Publication
(10) Pub. No.: US 2003/0017590 A1
(43) Pub. Date: Jan. 23, 2003

(54) PRODUCTION OF HUMAN CNS NEURONS FROM EMBRYONAL CARCINOMA CELLS USING CELL AGGREGATION METHOD

(76) Inventors: NANCY Y. IP, Hong Kong (HK); WILLIAM M.W. CHEUNG, Sunshine City (HK)

Correspondence Address: ROBERT D KATZ ESQ, COOPER & DUNHAM LLP, 1185 AVENUE OF THE AMERICAS, NEW YORK, NY, 10036

(21) Appl. No.: 09/436,084

(22) Filed: Nov. 8, 1999

(51) Int. Cl. C12N 5/08; C12N 5/00; C12N 5/02

(52) U.S. Cl. 435/368; 435/377; 435/402

(57) ABSTRACT

The invention provides a method for rapid production of pure CNS neurons from a cancer cell line comprising the steps of:

(a) replating an embryonic carcinoma cell line culture at a density of approximately 1×10⁵ cells per ml, and then allowing the culture to form cell aggregates by incubating the culture;

(b) adding all-trans retinoic acid to the cell culture containing cell aggregates to obtain an all trans retinoic acid concentration ranging from about 1 to about 20 μm

(c) incubating the culture until neurons are differentiated; and

(d) isolating the neurons from the culture.

The present invention concerns a novel methodology for the production of human CNS neurons.

Retinoic acid (RA), a derivative of vitamin A, induces differentiation in many cell types such as epithelial and cancer cells (Chambon, P., 1996, FASEB J. 10:940-954; De Luca, L. M., 1991, FASEB J. 5:2924-2933). Among the human cell types known to respond to RA, the human embryonal carcinoma (EC) cell line, NTera2 cl.D/1 (NT2), is one of the most extensively characterized (Andrews, P. W. et al., 1984, Lab. Invest. 50:147-162; Pleasure, S. J. and V. M. -Y. Lee, 1993, J. Neurosci. Res. 35:585-602). Following treatment with RA for four weeks, NT2 cells as monolayer cultures undergo terminal differentiation and become postmitotic CNS neurons (Andrews, P. W., 1984 Dev. Biol. 103:285-293). Successive replating of RA-treated NT2 cells in the presence of growth inhibitors result in the isolation of purified neurons (NT2-N) (Pleasure, S. J. et al., 1992, J. Neurosci. 12:1802-1815). These NT2-N neurons have been used as model systems for the study of neurodegenerative diseases such as Alzheimer's disease (Chyung, A. S. C. et al., 1997, J. Cell Biol. 138:671-680). The potential applications of NT2-N neurons in cell transplantation therapy for CNS disorders and their use as vehicles for delivering exogenous proteins into the human brain for gene therapy were recently demonstrated (Trojanowski, J. Q. et al., 1997, Exp. Neurol. 144:92-97).

When compared with murine EC cells such as P19, which are cultured as aggregates and are capable of differentiating into CNS neurons (Bain, G. et al., 1995, Dev. Biol. 168:342-357; Jones-Villeneuve, E. M. V. et al., 1983, Mol. Cell Biol. 3:2271-2279), NT2 cells require a relatively high concentration of RA (10 μM) to induce differentiation. Furthermore, the purification of NT2-N neurons involves a time-consuming process (˜eight weeks) and tedious primary culture techniques. The procedures of frequent dislodgment and replating might also result in the loss of neuron subtypes, such as those expressing tyrosine hydroxylase (TH) which are found in newly differentiated NT2-N neurons (lacovitti, L. and N. D. Stull, 1997, NeuroReport 8: 1471-1474). It is possible that the high concentration of RA and the lengthy period required for the differentiation of NT2 cells may partly be explained by the species difference between the two embryonal carcinoma cell lines, i.e., human versus murine. Alternatively, there exists the possibility that other extrinsic factors, such as cell-cell interaction, may play a critical role in the process of neuronal differentiation in NT2 cells.

The importance of cell aggregation generally defined herein as the accumulation of a large number of cells per unit volume) in initiating neuronal differentiation has been reported in other cell types such as embryonic stem cells and the P19 EC cells (Larue L., et al., 1996, Development 122:3185-94; Smith, S. C. et al., 1987, J. Cell. Physiol. 131:74-84). Since undifferentiated NT2 cells resemble neuroepithelial cells and retain properties of stem cells (Pleasure, S. J. and V. M. -Y. Lee, 1993, J. Neurosci. Res. 35:585-602), it is possible that cell-cell interaction can be enhanced by forming cell aggregates as in P19 cells (Jones-Villeneuve, E. M. V. et al., 1982, J. Cell Biol. 94:253-262).

It would be desirable, therefore to provide an improved technique for obtaining pure neuron cells from cancer cell lines which do not require lengthy primary culture techniques. It is therefore an object of the invention to provide a method for obtaining a pure CNS cell line using cell aggregate techniques.

### SUMMARY OF THE INVENTION

The foregoing disadvantages of the prior techniques are overcome by the methods and practices of the present invention, which provides an efficient methodology for the preparation of human CNS neurons.

Thus the present invention provides a method for the production of pure CNS neurons from a cancer cell line comprising the steps of:

(a) replating an embryonic carcinoma cell line culture at a density of approximately 1×10⁵ cells per ml, and then allowing the culture to form cell aggregates by incubating the culture;

(b) adding all-trans retinoic acid to the cell culture containing cell aggregates to obtain an all trans retinoic acid concentration ranging from about 1 to about 20 μM;

(c) incubating the culture until neurons are differentiated; and

(d) isolating the neurons from the culture.

### BRIEF DESCRIPTION OF THE DRAWINGS

The details of the invention will be further apparent from the following description with reference to the accompanying drawings, in which:

FIGS. 1(a)-(c) are bright field phase contrast photomicrographs (scale bar, 100 μm) cultured as aggregates with RA for 14 (RA14) or 18 (RA18) days. Control cells (FIG. 1(a)) were maintained as aggregates without RA for 14 days. Bright-field phase contrast photomicrographs scale bar, 100 μm of Scale bar, 100 μm.

FIG. 2 are NT2 neurons differentiated by cell aggregation expressed neuronal specific proteins, wherein NT2 cells were aggregated and differentiated by RA (5 μM) for 14 days, two days after replating and cultured in RA-free medium, differentiated NT2 cells were fixed and stained with the antibodies specific for neurofilament protein 200 kDa (NF200), synapsin I (synI), substance P (SP), and GABA.

FIG. 3 shows NT2 neurons differentiated by cell aggregation expressed tyrosine hydroxylase (TH). NT2 cells were aggregated and differentiated by RA (5 μM) for 14 days. Two days after replating in RA-free medium, differentiated NT2 cells were fixed and stained with the antibodies specific against TH (upper panel), and phosphorylated NF-M (lower panel). Scale bar, 50 μm.

FIG. 4 are bright field phase contrast photomicrographs (scale bar 500 μm) illustrating the effect of substratum on the neuronal differentiation of NT2 cells. NT2 aggregates were treated with RA (10 μM) for 14 days and replated onto 24-well plates precoated with either PDL, LAM or a combination of both. The cell aggregates were cultured in RA-free medium for 2 days.

FIG. 5. Expression of neuronal markers in RA differentiated NT2 aggregates. NT2 cells were pre-aggregated by RA at concentration (10⁻⁷ to 10⁻⁵ M) for 14 days and replated onto PDL/LAM coated plates for two days. As control experiments, NT2 cells were plated at 2×10⁵ cells/ml as monolayer cultures and differentiated with RA (10⁻⁷ to 10⁻⁵ M) for a total of 16 days. Total proteins were extracted and Western blot analysis was performed using antibodies specific against phosphorylated NF-M (NF-M-P; upper panel) and synapsin I (lower panel).

FIG. 6 are bright-field phase contrast photomicrographs (scale bar, 50 μm) illustration show aggregation reduced the duration of RA treatment required for the neuronal differentiation of NT2 cells. NT2 cells were treated as aggregates with RA (10 μM) for either 2, 5, or 14 days (RA2, RA5, RA14) followed by incubation in RA-free medium as aggregates for the remaining period (CTL12, CTL9, CTL0). The cell aggregates were plated onto PDL/LAM and further cultured for 2 days in the absence of RA.

### DETAILED DESCRIPTION OF THE PREFERRED EMBODIMENTS

As background to the invention, the basic molecular biological techniques employed in the present invention may be found in Freshney, R. I. et al., 1986, Animal Cell Culture: A Practical Approach, IRL Press, Oxford; Celis, J. E. et al., 1994, Cell Biology: A Laboratory Handbook, Academic Press, California; Sambrook, J. et al., 1989; and Molecular Cloning: a Laboratory Manual, Cold Spring Harbour Laboratory, New York.

Cell Culture

NT2 cells were cultured as previously described (Pleasure, S. J. et al., 1992, J. Neurosci. 12:1802-1815). Briefly, cells were maintained in OptiMEMI-reduced serum medium (GIBCO, Palo Alto, Calif., USA) supplemented with 5% fetal bovine serum (FBS; GIBCO, Palo Alto, Calif., USA). When NT2 cells were differentiated by the standard protocol, NT2 cells were seeded at 2×10⁶ cells per 75 cm² in Dulbecco's Modified Eagle's medium (high glucose formulation) (DMEM-HG; GIBCO, Palo Alto, Calif., USA) and were treated with a final concentration of 10 μM all-trans RA (SIGMA, St. Louis, Mo., USA). In the cell aggregation studies, cells were dislodged by trypsinization and were plated at 1×10⁵ cells per ml into a 85-mm bacteriological grade petri dish in DMEM-HG supplemented 10% FBS. After incubation overnight at 37° C. in a 5% CO₂ incubator, the aggregates were supplemented with a final concentration of 10 μM all-trans RA. The medium was replaced every two days. After 14 days of RA treatment, aggregates were plated onto tissue culture grade petri dish pre-coated with 10 μg/ml poly-D-lysine (PDL; SIGMA, St. Louis, Mo., USA), 10 μg/ml mouse laminin (LAM; GIBCO, Palo Alto, Calif., USA) and 0.1% gelatin (SIGMA, St. Louis, Mo., USA) in Dulbecco's Phosphate buffered saline (D-PBS; GIBCO, Palo Alto, Calif., USA). Following overnight incubation, aggregates were then cultured in DMEM-HG with 10% FBS supplemented with 1 μM cytosine D-arabinofuranoside (arac; SIGMA, St. Louis, Mo., USA) and 10 μM uridine (SIGMA, St. Louis, Mo., USA). After 3 days of incubation, neurons were detached by tapping gently on the side of the tissue culture plate, or by brief trypsinization, and replated on to a new pre-coated tissue culture plate. Incubation was continued in the presence of araC and uridine for another 4 days. Neurons obtained could be kept in DMEM supplemented with 10% FBS for more than 3 weeks.

Indirect Immunofluorescent Analysis

For immunofluorescent analysis, control or differentiated aggregates cultures were fixed with 4% paraformaldehyde (FLUKA, Hauppauge, N.Y., USA) in PBS. After blocking with 4% normal serum (NS; VECTOR LAB, Burlingame, Calif., USA) in PBS containing 0.4% Triton-X (TX; BOERINGER MANNEHEIM, Mannheim, Germany), and 1% bovine serum albumin (BSA; SIGMA, St. Louis, Mo., USA), aggregates were incubated overnight at 4° C. with primary antibodies diluted in antibody diluent (1% NS, 0.4% TX and 1% BSA). Incubation with secondary antibodies was performed at room temperature for at least 2 hours. Cells were visualized under fluorescence microscope (ZEISS, Jena, Germany). Antibodies used for the studies include polyclonal rabbit anti-200 kDa neurofilament protein (NF-H; CHEMICON, Temecula, Calif., USA), monoclonal mouse anti-phosphorylated NF-M protein (NF-M-P; ZYMED, South San Francisco, Calif., USA), polyclonal rabbit anti-substance P (SP; CHEMICON, Temecula, Calif., USA), polyclonal rabbit anti-GABA (SIGMA, St. Louis, Mo., USA), and polyclonal rabbit antityrosine hydroxylase (TH; CHEMICON, Temecula, Calif., USA). Mouse synapsin I antibodies (a generous gift from A. Baines) were used to detect presynaptic specialization. FITC-conjugated goat anti-rabbit IgG (CAPPEL, Turnhout, Belgium) and rhodamine-conjugated goat anti-mouse IgG (CAPPEL, Turnhout, Belgium) were used as secondary antibodies.

Total Protein Extraction and Western Blot Analysis

Cells were washed twice with ice-cold PBS followed by a wash with ice-cold PBS supplemented with 1 mM sodium orthovanadate (NaOV; SIGMA, St. Louis, Mo., USA). Cells were then lysed with lysis buffer (50 mM Tris.Cl at pH8, 150 mM NaCl, 1% TX, 1×aprotinin; SIGMA, St. Louis, Mo., USA, 1 mM PMSF; GIBCO, Palo Alto, Calif., USA and 1 mM NaOV) at 4° C. for 15 minutes. Lysates were collected and centrifuged to remove cell debris. Protein assays were performed with Bio-Rad Protein Assay Kit based on the Bradford dye-binding procedure (BIO-RAD, Hercules, Calif., USA). Protein (typically 10 μg) were separated by SDS-PAGE and then electro-transferred to nitrocellulose membrane (MSI, Westborough, Mass., USA). After blocking at room temperature for 1 hr using TBS-Tween containing 5% non-fat milk, membranes were incubated overnight at 4° C. with primary antibodies in TBS-Tween containing 5% non-fat milk. Membranes were then incubated with horseradish peroxidase conjugated secondary antibodies (1:2000 dilution; AMERSHAM, Arlington Heights, Ill., USA) for 1 hour at room temperature. After three washes with TBS-Tween, immunoreactive bands were detected using ECL kit (AMERSHAM, Arlington Heights, Ill., USA).

### EXAMPLE I

Cell Aggregation Reduced the Time Required to Form NT2 Neurons

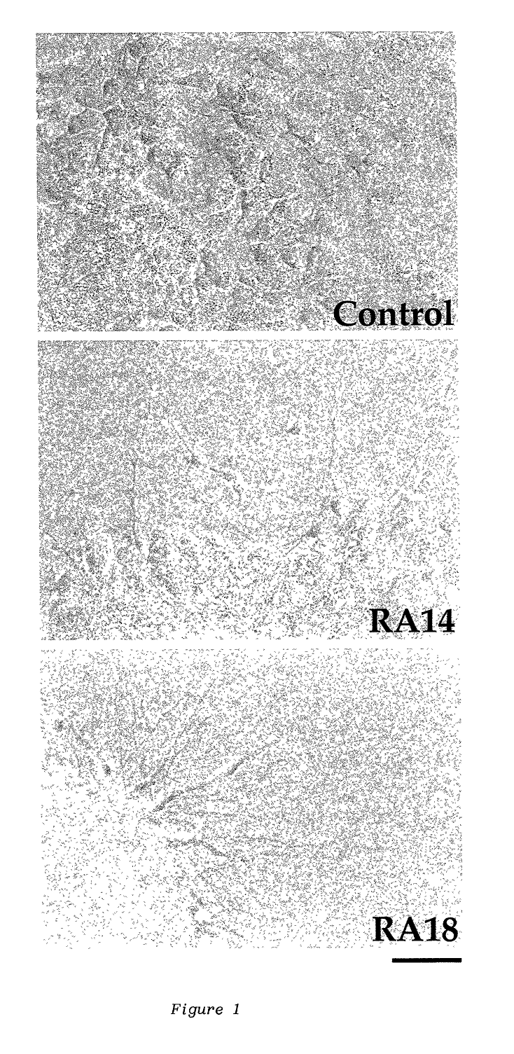

To examine the effect of cell aggregation on RA-induced neuronal differentiation, NT2 cells were pre-aggregated as described above. After 14 days of RA treatment, aggregates were plated on PDL and LAM (PDL/LAM) coated plates. Neurite outgrowth was initiated after overnight incubation in the absence of RA. Phase bright neuron-like cells with long extended neurites continued to migrate out from the NT2 aggregates. At day 3 after replating, these cells grew on the surface of large-flattened cells (FIG. 1). If NT2 aggregates were treated with RA for only 12 days, the extension of neurites from NT2 cells was observed 2 days after being plated onto PDL/LAM coated plates. However, neuron-like cells were subsequently overwhelmed by both undifferentiated NT2 cells and the differentiated large-flattened cells (Pleasure, S. J. et al., 1992, J. Neurosci. 12:1802-1815). Further reduction of the cell aggregation period to less than 12 days did not result in observable neuritic outgrowth RA-treated aggregates.

### EXAMPLE II

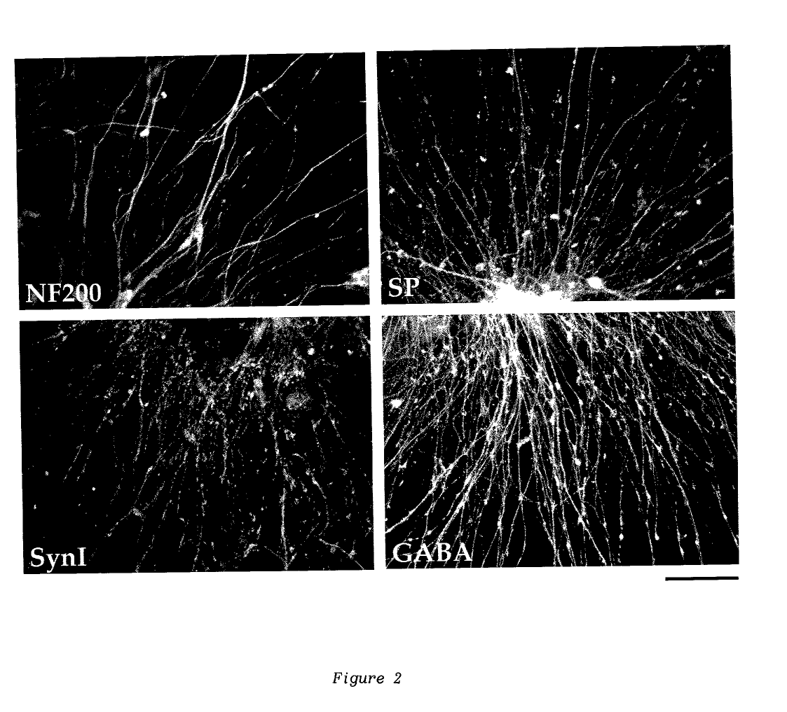

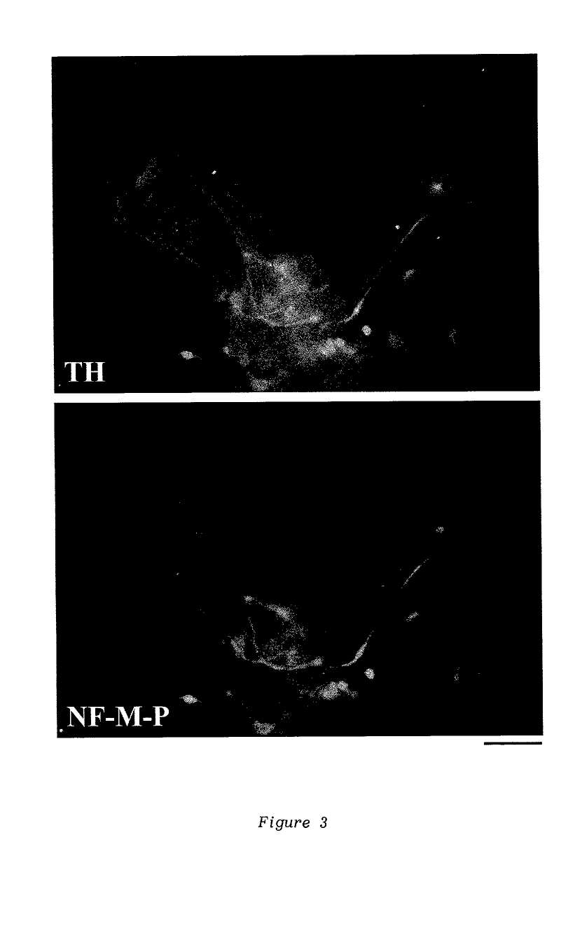

In order to verify whether the phase-bright cells obtained following cell aggregation were neurons, immunocytochemical analysis was performed. High-molecular weight neurofilament proteins (200 kDa) were detected in the neurites of the neurons (FIG. 2). Presynaptic specialization of the differentiated NT2 neurons was demonstrated by positive staining of the neuron-specific synapsin I in the varicosities of the neurites and in the cell body (FIG. 2). We have also examined the expression of neurotransmitters, such as GABA. Detection of GABA in these neurons (FIG. 2) demonstrated the presence of GABA-ergic neurons in the differentiated NT2 aggregates. In addition, substance P (SP) was detected in both the cell bodies and the neurites of the neurons (FIG. 2). TH-expressing neurons were also found in enriched NT2 neurons prepared using cell aggregation method (FIG. 3).

### EXAMPLE III

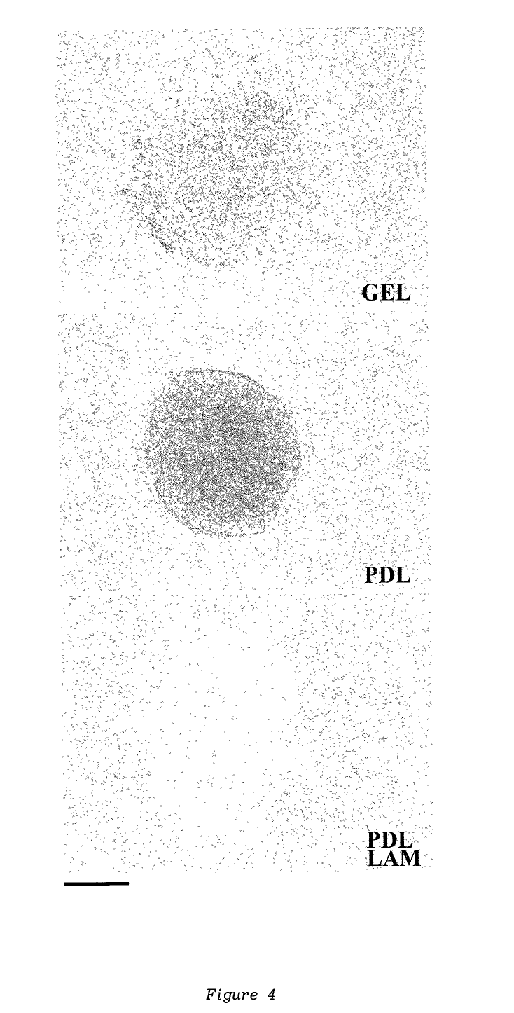

Neuronal differentiation of NT2 cells maintained as monolayer cultures was previously demonstrated to be influenced by the type of substrates (Pleasure, S. J. and V. M. -Y. Lee, 1993, J. Neurosci. Res. 35:585-602). We have compared the effects of various extracellular matrices on the differentiation of NT2 aggregates. After 14 days of RA treatment, NT2 aggregates were plated onto tissue culture plates coated either with gelatin, PDL, LAM alone or in combination (FIG. 4). Although gelatin and PDL allowed better attachment of the aggregates onto the plates, the use of PDL alone or PDL in combination with gelatin as substrates did not result in extension of neurites even after incubation of NT2 cells for more than 1 week. The presence of LAM or in combination with gelatin and PDL resulted in extension of neurites. Thus, extension of neurites from NT2 aggregates was dependent on the extracellular substrates.

### EXAMPLE IV

To study whether the high concentrations of RA required for inducing neuronal differentiation of NT2 cells could be lowered under the condition of cell aggregation, we exposed the NT2 aggregates to reduced concentration of RA (0.1 to 10 μM). Following treatment of NT2 aggregates with RA for 14 days, these cells were plated onto PDL/LAM coated plates in RA-free medium, and total protein samples were prepared 2 days later. As control, total protein samples were extracted from monolayer NT2 cultures treated with 16 days of RA (0.1 to 10 μM).

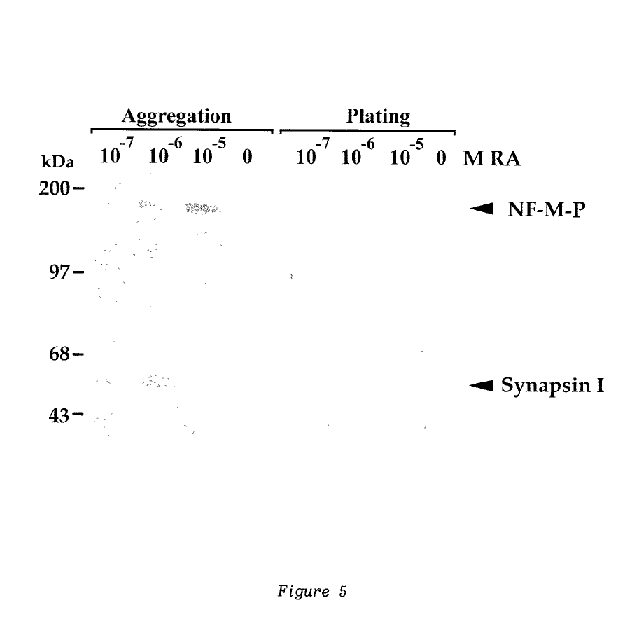

RA treatment at all concentrations (0.1 to 10 μM) examined could induce the expression of phosphorylated NF-M proteins in NT2 aggregates. On the other hand, monolayer NT2 cells expressed phosphorylated NF-M only at higher concentrations of RA (1 or 10 μM). The expression level of phosphorylated NF-M proteins in NT2 aggregates was elevated with increased concentration of RA used. A comparable expression level of phosphorylated NF-M proteins was detected when NT2 aggregates and monolayer NT2 cells were treated with 0.1 and 10 μM RA, respectively (FIG. 5, upper panel). This finding suggests that cell aggregation promotes the necessary biochemical changes that are important for the neuronal differentiation of NT2 cells and therefore allows RA to induce neurofilament protein expression even at reduced concentrations.

Similar results were obtained when the expression of synapsin I proteins in RA-treated NT2 aggregates were examined (FIG. 5, lower panel). High level of synapsin I protein expression was detected in RA-treated (0.1 to 10 μM) NT2 aggregates but not in monolayer NT2 cell cultures. It should be noted that the number of neurons that could be prepared diminished with reduced concentration of RA, i.e. only a few neurons could be observed at 1 μM RA while almost no neurons were observed when the RA concentration was further reduced to 0.1 μM (data not shown). In the absence of RA treatment, aggregation alone could not result in any observable neuronal phenotype.

### EXAMPLE V

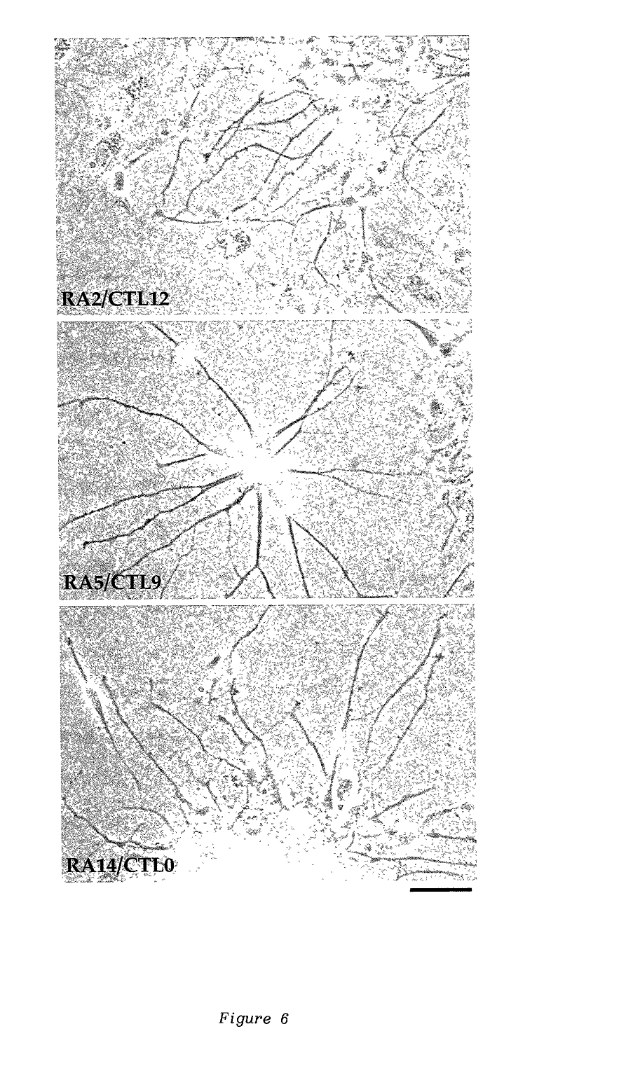

Under monolayer culture conditions, a brief period of RA treatment resulted in changes in cell morphology without the formation of NT2 neurons; continuous RA treatment of NT2 cells is therefore essential for the neuronal differentiation of NT2 cells (Andrews, P. W., 1984 Dev. Biol. 103:285-293). It is possible that the sustained requirement of RA for the neuronal differentiation of NT2 is indeed due to the lack of cell-cell interaction. To examine this possibility, NT2 cells were maintained as aggregates in bacteriological culture dish for 14 days and RA treatment was given only during the first few days of aggregation (1 to 5 days). Neurons were observed following only 2 to 3 days of RA treatment, provided NT2 cells were maintained as aggregates for 14 days (FIG. 6).

### DISCUSSION

NT2 cells have been extensively used as in vitro model systems for various studies related to cancer therapy, CNS development, and neurodegenerative diseases. However, the strategy used for the preparation of these NT2 neurons required lengthy RA treatment and might result in the loss of specific sub-populations of neurons. We aimed at developing a rapid approach to produce NT2 neurons by introducing other extrinsic factors, such as cell-cell interaction, that are essential during the process of neuronal differentiation. In the present study, we report that when NT2 cells were treated with RA while growing as cell aggregates in bacteriological plates, neurons were formed within 16 days. Using the standard protocol for differentiation, neuron-like cells could only be observed about five weeks after the RA treatment (Pleasure, S. J. et al., 1992, J. Neurosci. 12:1802-1815). In our study, promotion of neuronal differentiation by cell aggregation is also revealed by the higher level of expression of phosphorylated NF-M proteins and the early expression of the synapsin proteins in RA-treated NT2 aggregates.

While cell aggregation can promote neuronal differentiation of NT2 cells, our findings suggest that it could not replace RA as an extrinsic factor to trigger the process of neuronal differentiation. In the absence of RA, there was no observable neuronal phenotype in NT2 aggregates despite very low level of phosphorylated NF-M expression. A low concentration of RA (0.1 μM) was enough to induce the expression of phosphorylated NF-M in NT2 aggregates but did not result in cells that resembled neurons. Thus, under cell aggregation conditions, lower concentration of RA is enough to induce neuron-related biochemical changes of the NT2 cells, albeit not enough for the presentation of the neuronal phenotypes. Taken together, our findings suggest that RA serves as a decisive signal that initiate the biochemical changes involved in the neuronal differentiation process, such as the expression of a threshold level of neurofilament proteins. However, the presentation of the neuronal phenotypes is facilitated by the other extrinsic factors, such as the cell-cell interaction provided by the cell aggregation in this study.

It should be noted that the period (=14 days) required for the maintenance of NT2 cells as aggregates is critical. This important experimental paradigm may well account for the inability of a previous study on NT2 cells to demonstrate any significant effects of cell aggregation on the formation of neurons (Andrews, P. W., 1984 Dev. Biol. 103:285-293). In our study, we have demonstrated that provided the cell aggregation process was long enough (=14 days), RA treatment could be reduced (3 to 5 days). It is possible that RA serves to activate the pathways leading to the expression of genes necessary for the neuronal differentiation and that the maintenance of such expression is regulated either by the presence of RA or by the ‘factors’ produced as a consequence of cell aggregation. Candidates for the genes regulated by RA include the neurotrophin receptors and their cognate ligands and novel sequences (Cheung, W. M. W. et al., 1996, NeuroReport 7:1204-1208; Cheung W. M. W. et al., 1997, J. Neurochem. 68:1882-8).

The molecular mechanism underlying the effects of aggregation remains to be elucidated. It is possible that aggregation mimics the anti-proliferative effect of RA during the neuronal differentiation of NT2 cells, similar to what was previously demonstrated in immortalized neuroectodermal precursor cells (Toht, M. et at., 1995, J. Neurosci. Res. 41:764-774). Alternatively, aggregation might induce the synthesis of RA in situ, thereby reducing the time required for RA treatment. Studies are in progress in our laboratory to elucidate the mechanism involved in the actions of aggregation during the RA-induced neuronal differentiation of NT2 cells. One useful strategy is to perform extensive transcripts analysis to isolate candidate genes that might be involved in the aggregation process. This type of approach has proved to be useful in the P19 system (Marazzi, G. et al., 1997, Dev. Biol. 186:127-138). Further studies are needed to provide a better understanding on the role of aggregation in promoting neuronal differentiation of NT2 cells, as well as on the interaction between extrinsic factors that influence the ultimate cell fate of a neuronal precursor cell.

NT2 neurons have been used extensively in studies aimed at understanding the mechanisms that underlie neuronal degenerative diseases such as Alzheimer's disease. For example, expression of specific forms of acetycholinesterase in NT2 cells allows the elucidation of the roles of these enzymes which are selectively lost in Alzheimer's patients (Llanes, C. et al., 1995, J. Neurosci. Res. 42:791-802). Moreover, NT2 neurons provide a rich source of neurons for use in cell transplantation therapy which has recently been suggested to be one of the most promising treatment strategies for various neurodegnerative diseases (Hoffer, B., and L. Olson, 1997, J. Neural Transmission Suppl. 49:1-10). Transplantation of neurons into the CNS lesioned site has resulted in the restoration of the normal nigrostriatal pathway in patients suffering from Parkinson's disease (Trojanowski, J. Q. et al., 1997, Exp. Neurol. 144:92-97). Thus, the rapid protocol described in the present study will be useful in providing an efficient way to produce large quantities of neurons for basic research as well as applications in cell therapy. Moreover, these transfectable postmitotic CNS neurons can also be used to deliver gene products directly into specific areas of the diseased human CNS.

What is claimed is:

1. A method of the production of pure CNS neurons from a cancer cell line, comprising:
(a) replating an embryonic carcinoma cell line culture at a density of approximately 1×10⁵ cells per ml, and then allowing the culture to form cell aggregates by incubating the culture;
(b) adding all-trans retinoic acid to the cell culture containing cell aggregates to obtain an all trans retinoic acid concentration ranging from about 1 to about 20 μM;
(c) incubating the culture until neurons are differentiated; and
(d) isolating the neurons from the culture.

2. A method in accordance with claim 1 wherein the embryonic carcinoma cell line is Ntera2.cl.D/1 (NT2).

3. A method in accordance with claim 2 wherein the culture is incubated in the presence of all trans retinoic acid for at least about twelve days.

4. A method in accordance with claim 3, wherein the incubating in step (c) takes place in a culture dish precoated with 1 to 20 μg/ml poly-D-lysine, 1 to 20 μg/ml mouse laminin and 0.1 to 0.4% w/v gelatin.

5. A method in accordance with claim 1 where the all trans retinoic acid concentration is about 10 μM.

6. A method in accordance with claim 4, wherein the incubating in step (c) takes place in a culture dish precoated with about 10 μg/ml poly-D-lysine and about 10 μg/ml mouse laminin.
